(19) United States
(12) Patent Application Publication
(10) Pub. No.: US 2024/0075173 A1
(43) Pub. Date: Mar. 7, 2024

(54) MERCHANDISERS WITH A PACKAGE SANITIZATION SYSTEM

(71) Applicant: THE COCA-COLA COMPANY, Atlanta, GA (US)

(72) Inventors: Candace Nicola WILSON, Decatur, GA (US); Camila DA ROSA LOPES, Woodstock, GA (US); Marcelo TOSAKI, Lawrenceville, GA (US); Alan Lee HAWKINS, Conyers, GA (US); Venkat GOVINDARAJAN, Duluth, GA (US)

(21) Appl. No.: 18/262,215

(22) PCT Filed: Feb. 2, 2022
(86) PCT No.: PCT/US2022/014904

§ 371 Date: Jul. 20, 2023

Related U.S. Application Data
(60) Provisional application No. 63/145,060, filed on Feb. 3, 2021.

(51) Int. Cl. A61L 2/10; A61L 9/20

(52) U.S. Cl. CPC A61L 2/10; A61L 9/20; A61L 2202/11; A61L 2202/122; A61L 2202/14; A61L 2202/23; A61L 2209/12

(57) ABSTRACT

The present application provides a merchandiser with a number of packages therein. The merchandiser may include a product area with either a number of shelves or a number of columns with the packages therein and a package sanitation system. The package sanitation system may include one or more ultraviolet lights positioned within the product area so as to sanitize the packages therein.

### TECHNICAL FIELD

The present application and the resultant patent relate generally to merchandisers and more particularly relate to merchandizers such as vending machines, coolers, and the like with a package sanitization system to safely maintain and dispense the packages therein.

### BACKGROUND OF THE INVENTION

Consumers are increasingly concerned with package safety and integrity for food and beverage products. Specifically, consumers are concerned with how packages are handled and whether the package surface needs to be cleaned or disinfected. Such consumer concerns may be lessened if the packages could be sterilized before being dispensed from, for example, a merchandiser such as a vending machine, a cooler, and the like.

There are a number of known decontamination techniques, each with different benefits and drawbacks. For example, there is wet decontamination with different types of liquid chemicals, dry decontamination through chemical means or energy radiation, and the use of active agents that prevent microbial growth on the package surface. Not all of these techniques, however, are applicable or even practical for sanitizing the packages in a merchandiser and other types of package enclosures in a safe and efficient manner.

There is thus a desire for an improved merchandiser or other type of enclosure with a safe and effective package sanitation system. Such a package sanitation system may maintain and dispense the packages therein in a safe and hygienic manner. Moreover, such a package sanitation system may inform the consumer that the package is safe to handle.

### SUMMARY OF THE INVENTION

The present application and the resultant patent thus provide a merchandiser with a number of packages therein. The merchandiser may include a product area with either a number of shelves or a number of columns with the packages therein and a package sanitation system. The package sanitation system may include one or more ultraviolet lights positioned within the product area so as to sanitize the packages therein.

The present application and the resultant patent further provide a method of sanitizing packages within a merchandiser. The method may include the steps of turning on an ultraviolet light within the merchandiser for a predetermined amount of time, determining when a door of the merchandiser has been opened, and again turning on the ultraviolet light for the predetermined amount of time upon the determination that the door of the merchandiser has been opened.

The present application and the resultant patent further provide a merchandiser with a number of packages therein. The merchandiser may include either a number of shelves or a number of columns with the packages therein and a package sanitation system. The package sanitation system may include a number of ultraviolet lights positioned about the shelves or the columns so as to sanitize the packages therein.

These and other features and improvements of the present application and the resultant patent will become apparent to one of ordinary skill in the art upon review of the following detailed description when taken in conjunction with the shown drawings and the appended claims.

### BRIEF DESCRIPTION OF THE DRAWINGS

FIG. 1 is a schematic view of a merchandiser.

FIG. 2 is a side view of a merchandiser as a cooler with a package sanitation system as may be described herein.

NI HI FIG. 3 is a front view of the cooler of FIG. 2.

FIG. 4 is a side view of a shelf with packages thereon in the cooler of FIG. 2.

FIG. 5 is a front view of the shelf with packages thereon of FIG. 4.

FIG. 6 is an expanded perspective view of a bottom exit of the shelf of FIG. 4.

FIG. 7 is an expanded top view of the bottom exit of the shelf of FIG. 4.

FIG. 8 is a flow chart showing exemplary steps in a timing algorithm of the package sanitation system of FIG. 2.

FIG. 9 is a front view of a merchandiser as a vending machine with a package sanitation system as may be described herein.

FIG. 10 is an expanded side view of the vending machine with the package sanitation system of FIG. 9.

FIG. 11 is an expanded side view of the vending machine with the package sanitation system of FIG. 9.

### DETAILED DESCRIPTION

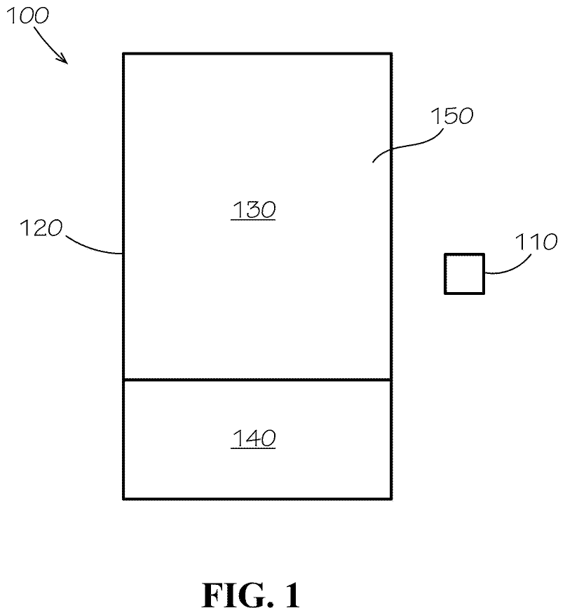

Referring now to the drawings in which like numbers refer to like elements throughout the several views, FIG. 1 shows an example of a merchandiser 100 as may be described herein. The merchandiser 100 may have any number of product packages 110 therein. The merchandiser 100 may include an outer shell 120. In this example, the outer shell 120 may have a substantially rectangular configuration, but the outer shell 120 may have any suitable size, shape, or configuration. The outer shell 120 may be made out of metals, plastics, or any suitable rigid material. The outer shell 120 may be insulated in whole or in part. The outer shell 120 may define a product area 130 and a refrigeration component area 140. The refrigeration component area 140 may include conventional refrigeration and/or heating components and the like therein. The outer shell 120 may include an access door 150. The access door 150 may have any suitable size, shape, or configuration. Other components and other configurations also may be used herein.

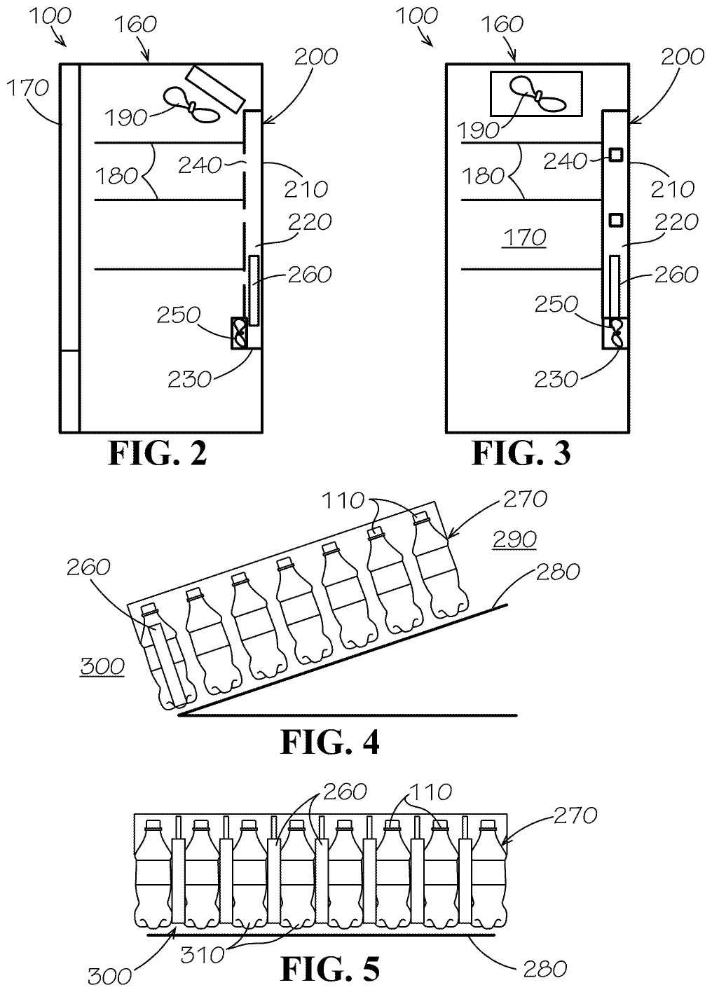

FIGS. 2 and 3 show an example of a merchandiser 100 as a cooler 160. As is known, the cooler 160 may include the access door 150 being a glass front door 170 so as to allow the consumer to view the packages 110 therein. The cooler 160 may have a number of shelves 180 therein so as to support the packages 110. Any number of shelves 180 may be used herein in any size, shape, or configuration. The product area 130 may have one or more refrigeration fans 190 and the like in communication with the refrigeration component area 140. The refrigeration fans 160 may be of conventional design. In use, the consumer simply opens the glass front door 170 and selects one of the packages 110 on the shelves 180. Other components and other configurations may be used herein.

The cooler 160 may include a package sanitation system 200 positioned thereon. The package sanitation system 200 may include a number of alternative configurations. In the example of FIGS. 2 and 3, the package sanitation system 200 may be in the form of an air recirculation sanitation system 210. The air recirculation sanitation system 210 may include an air duct 220 positioned with the product area 130. The air duct 220 may have an entrance 230 and one or more exits 240 positioned about the shelves 180. The air duct 220 may have any suitable size, shape, or configuration. The air recirculation system 210 may have a recirculation fan 250 positioned about the entrance 230 of the air duct 220 or elsewhere. The recirculation fan 250 may be a low CFM axial flow fan and the like. The air recirculation sanitation system 210 also may have an ultraviolet (UV) light source 260 position within the air duct 220. As will be described in more detail below, the UV light source 260 may operate in certain wavelengths to inactivate microorganisms and the like. Other components and other configurations may be used herein.

In use, the recirculation fan 250 may move the air inside the cooler 160 through the air duct 220. The air will pass by the UV light 260 within a certain proximity so as to eliminate airborne bacteria and the like that may enter while the glass front 170 door is open. Positioning the UV light 260 inside the air duct 220 also protects consumers from potential exposure to the UV light. After each door open/close event, the recirculation fan 250 may turn on and run long enough to sanitize the volume of air inside the cooler 160. The air duct 220 may be installed to direct the air flow in the manner deemed most effective in reducing contamination. Other components and other configurations may be used herein.

FIGS. 4 and 5 show a further example of a package sanitation system 200 for coolers 160. This example may show a product shelf sanitation system 270. Specifically, the shelves 180 within the cooler 160 may be in the form of gravity fed shelves 280 positioned at an angle such that the packages 110 may move under the force of gravity from a top entrance 290 to a bottom exit 300. Each shelf 180 also may be divided into a number of rows 310 so as to control the advance of each package 110 therein. Any number of the rows 310 may be used herein. Other components and other configurations may be used herein.

The product shelf sanitation system 270 may include a pair of UV lights 260 about the bottom exit 300 of each row 310 or elsewhere. Each UV light 260 of the pair thus covers one half of the package 110 with emitted UV light such that the vertical surfaces are covered in full by the emitted UV light. The product shelf sanitation system 270 thus sanitizes each package 110 just before removal from the row 310 and the cooler 160.

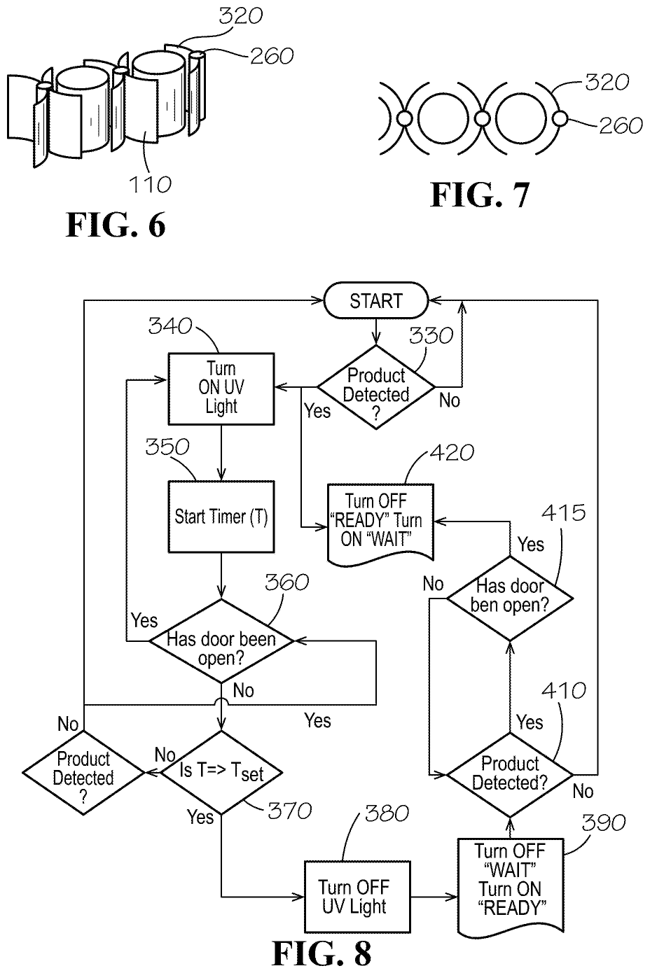

FIGS. 6 and 7 show a further embodiment of the product shelf sanitation system 270. In this example, the bottom exit 300 of each row 310 also may include a pair of deflectors 320 positioned about the UV lights 260. The deflectors 320 may concentrate the emitted UV light and prevent the UV light from being shed in unwanted directions. Other components and other configurations may be used herein.

The product shelf sanitation system 270 also may limit the extent of UV exposure. Specifically, the UV lights 260 may be timed and switched off in order to prevent product deterioration and/or packaging breakdown due to overexposure to the emitted UV light. Also, the time of exposure to the UV light may optimize sanitization and product preservation. The exposure time (T) is a variable that can be set in the overall cooler controller or elsewhere.

FIG. 8 shows an exemplary flow chart for the operation of the UV lights 260 based on the opening of the opening of the door 170 of the cooler 160. Specifically, the decontamination process may be reinitialized every time the door 170 is open, because the previous package decontamination process may have been compromised during the time the door 170 was open.

In step 330, the process is initiated and it is determined if there is a package 110 in the bottom exit 300 of a given row 310. If so, the UV lights 260 are turned on at step 340 and the timer is started at step 350. At step 360, it is determined if the door 170 has been opened. If not, it is determined at step 370 if the elapsed time T is greater than or equal to a set time, Tₛₑₜ. If so, the UV lights 260 are turned off at step 380. At step 390, system status is changed from “WAIT” to “READY.” At step 400, it is again determined if there is a package 110 detected in the bottom exit 300 of a given row 310. If so, it is determined at step 415 if the door 170 has been opened. If so, system status is changed from “READY” to “WAIT” at step 420. The process then returns to step 340 where the UV lights 260 are again turned on. Depending upon the nature of the cooler 160, a signal or other type of indicia may indicate to the consumer that the package 110 has been sanitized. The steps described herein are exemplary only. Many different and other steps may be used herein in any order.

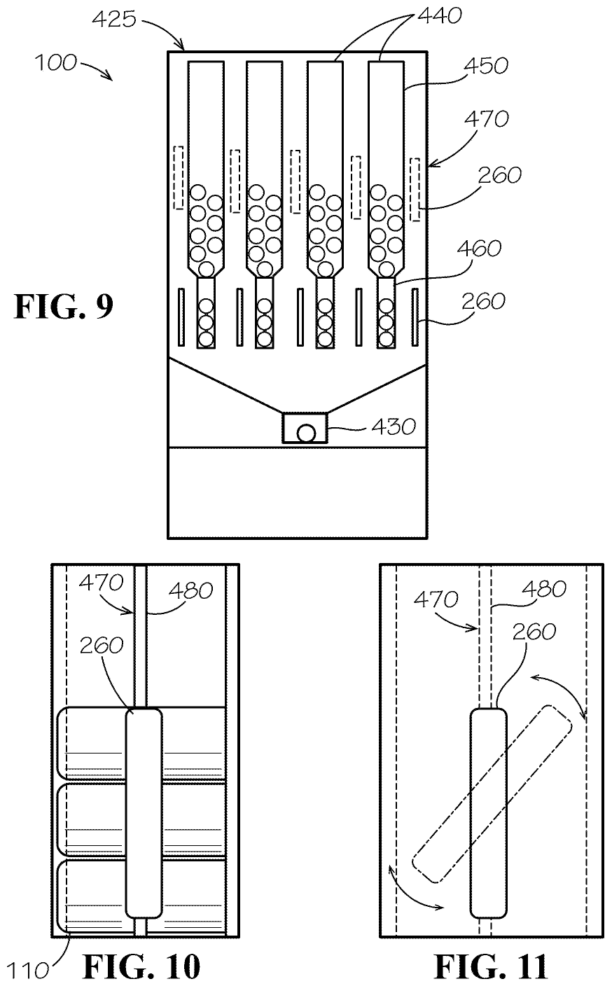

FIGS. 9-11 show a further example of a merchandiser 100. In this example, the merchandiser 100 may be in the form of a vending machine 425. As is known, the vending machine 425 may include a vending port 430 on the access door 150 so as to allow the consumer to access a package 110 vended therein. The vending machine 425 may have a number of internal vending columns 440 therein in communication with the vending port 430. The vending columns 440 may have an upper stack area 450 and a lower pre-vend area 460. Any number of the vending columns 440 may be used herein in any suitable size, shape, or configuration. Other components and other configurations may be used herein.

The vending machine 425 also may include the package sanitation system 200 positioned therein. In this example, the package sanitation system 200 may be in the form of vending column sanitation system 470. The vending column sanitation system 470 may include a number of the UV lights 260 positioned about each of the vending columns 440. For example, a pair of the UV lights 260 may be positioned about the upper stack area 450 and a pair of the UV lights 260 may be positioned about the lower pre-vend area 460. Other positions may be used herein. The UV lights 260 positioned about the upper stack area 450 may sanitize the packages 110 as they are loaded into the vending machine 425 while the UV lights 260 positioned about the pre-vend area 460 may sanitize the packages 110 before or while being dispensed. Other components and other configurations may be used herein.

As is shown in FIG. 10, the UV lights 260 may be positioned about the vending columns 440 on the metal guide bars 480 therein so as to have good exposure to the packages 110 therein. As is shown in FIG. 11, the UV lights 260 may be movable. Specifically, the UV lights 260 may rotate and/or move up and down the vending columns 440 so as to increase exposure of the packages 110 to the emitted UV light. A timing algorithm similar to that described above may be used herein. Other components and other configurations may be used herein.

Different types of UV light sources may be used herein. Shortwave ultraviolet light or radiation (UV-C) has been used successfully for fresh produce decontamination. UV-C radiation is a nonthermal/nonchemical intervention technology that employs physical light energy of a specific wavelength to inactivate microorganisms. The germicidal effect of UV-C light (i.e., UV between about 200 and 290 nm) is a result of its ability to damage the DNA or RNA of a microorganism. UV-C light can effectively kill microorganisms including pathogenic bacteria and viruses. Treatment with UV-C technology offers several advantages given that it does not leave chemical residue or require extensive protection equipment. Microorganisms must be directly exposed to UV-C light to be inactivated. Therefore, ensuring uniform exposure of UV-C light to all surfaces is a technical challenge. Utilizing multiple UV lights 260 may help provide more uniform UV-C exposure from multiple directions and angles to the UV-C light.

To measure accurately the UV-C doses that each package 110 receives, a dosimetry system that can indicate doses on different locations of the package surfaces may be used. For example, a plastic film with embedded dye that undergoes a color change when exposed to ionizing irradiation may be used. These colorless and transparent films are thin, strong, and flexible with good optical qualities. These films thus may be used as labels that provide consumers with some indication of the sanitization process.

Pulsed light (PL) technology also can be used to inactivate several pathogenic and spoilage microorganisms with a minimal impact on the quality attributes. The short duration—high power light pulses may inactivate microorganisms by a combination of photochemical, photothermal, and photophysical mechanisms. In addition to the inactivation of microorganisms, PL treatment has been shown to be effective in some in-package microbial decontamination, reduction in allergens, and for extending the shelf-life of certain foods while retaining its nutritional value. This technology also is known by several other names such as high-intensity light, broad-spectrum white light, intense pulsed light, pulsed white light, and pulsed UV light, and the like, with the majority of energy of pulsed light coming from the UV portion of the spectrum. PL systems produce broad spectrum light at wavelengths ranging from ultraviolet (UV) to near infrared (NIR, 100-1000 nm; UV [100-400 nm], visible light [380-780 nm], and infrared [700-1100 nm]). The light used for food processing applications typically are pulsed at 1-20 flashes per second and at an energy density in the range of 0.01-50 J/cm2 at the surface.

Using PL for sterilization provides numerous advantages over other methods: (1) increased safety; (2) small temperature changes that cause less damage to products; (3) short irradiation durations and high operational efficiency; and (4) possible uses in food manufacturing and processing as necessary. PL irradiation reduces microbial contamination and has the potential to extend the shelf life of products.

The systems and methods described herein thus provide improved package sanitation. Such improved sanitation may provide the consumer with reassurance that the package is safe to handle and that the product therein is safe to consume. Such systems and methods also may be combined with other types of vending and dispensing techniques such as touchless vending and the like so as to further enhance the consumer's confidence herein.

It should be apparent that the foregoing relates only to certain embodiments of the present application and the resultant patent. Numerous changes and modifications may be made herein by one of ordinary skill in the art without departing from the general spirit and scope of the invention as defined by the following claims and the equivalents thereof

We claim:

1. A merchandiser with a number of packages therein, comprising:
a product area;
the product area comprising at least one of a plurality of shelves or a plurality of columns with the number of packages therein; and
a package sanitation system;
the package sanitation system comprising one or more ultraviolet lights positioned within the product area so as to sanitize the number of packages.

2. The merchandiser of claim 1, wherein the merchandiser comprises a cooler.

3. The merchandiser of claim 2, wherein the package sanitation system comprises an air duct and a recirculation fan positioned within the product area of the cooler.

4. The merchandiser of claim 3, wherein the one or more ultraviolet lights are positioned about the air duct to sanitize an airflow therethrough.

5. The merchandiser of claim 2, wherein the cooler comprises a plurality of shelves therein.

6. The merchandiser of claim 5, wherein the plurality of shelves comprises a plurality of gravity fed shelves extending at an angle from a top entrance to a bottom exit.

7. The merchandiser of claim 6, wherein a pair of the one or more ultraviolet lights is positioned about bottom exit.

8. The merchandiser of claim 7, wherein the bottom exit comprises one or more deflectors positioned about each of the one or more ultraviolet lights.

9. The merchandiser of claim 1, wherein the merchandiser comprises a vending machine.

10. The merchandiser of claim 9, wherein the one or more ultraviolet lights are positioned about the plurality of columns.

11. The merchandiser of claim 10, wherein each of the plurality of columns comprises and upper stack area and a lower pre-vend area.

12. The merchandiser of claim 11, wherein the one or more ultraviolet lights are positioned about the upper stack area.

13. The merchandiser of claim 11, wherein the one or more ultraviolet lights are positioned about the lower pre-vend area.

14. The merchandiser of claim 1, wherein the one or more ultraviolet lights are maneuverable within the product area.

15. A method of sanitizing packages within a merchandiser, comprising:
turning on an ultraviolet light within the merchandiser for a predetermined amount of time;
determining when a door of the merchandiser has been opened; and
again turning on the ultraviolet light for the predetermined amount of time upon the determination that the door of the merchandiser has been opened.
